(19) United States
(12) Patent Application Publication
(10) Pub. No.: US 2021/0371865 A1
(43) Pub. Date: Dec. 2, 2021

(54) TLR9 AGONISTS FOR USE IN DOWNREGULATING CLEVER-I EXPRESSION ON ALTERNATIVELY ACTIVATED MACROPHAGES

(71) Applicant: FARON PHARMACEUTICALS OY, Turku (FI)

(72) Inventors: Maija-Leena HOLLMÉN, Piispanristi (FI); Miro VIITALA, Turku (FI)

(73) Assignee: FARON PHARMACEUTICALS OY, Turku (FI)

(21) Appl. No.: 17/285,868

(22) PCT Filed: Nov. 1, 2019
(86) PCT No.: PCT/FI2019/050778

§ 371 Date: Apr. 15, 2021

(30) Foreign Application Priority Data
Nov. 1, 2018 (FI) 20185926

(51) Int. Cl. C12N 15/117; A61P 35/00

(52) U.S. Cl. CPC C12N 15/117; C12N 2310/17; A61P 35/00

(57) ABSTRACT

The invention relates to use of TLR9 antagonist in a method for treating cancer and/or chronic infection by downregulating Clever-1 expression on alternatively activated immunosuppressive macrophages.

### FIELD OF THE INVENTION

The present invention relates generally to compositions and methods for treating cancers and/or chronic infections by using TLR9 agonists.

### BACKGROUND OF THE INVENTION

The publications and other materials used herein to illuminate the background of the invention, and in particular, cases to provide additional details the practice, are incorporated by reference.

The human Clever-1 (Common Lymphatic Endothelial and Vascular Endothelial Receptor-1), is known and it is disclosed in the publication WO 03/057130. Clever-1 is also known as Stabilin-1 or Feel-1. Clever-1 has also been reviewed by Kzhyshkowska J. (2010), TheScientificWorldJOURNAL 10, 2039-2053,“Multifunctional receptor Stabilin-1 in homeostasis and disease”. Clever-1 is expressed in lymphatic endothelial cells, certain vascular endothelial cells, but also alternatively activated immunosuppressive macrophages, often referred to as tumour associated macrophages. It is also previously presented in the publication WO2010/122217 that blocking of Clever-1 by Clever-1 specific antibodies reduce the size of malignant tumour and/or malignant tumour growth. It is also previously presented e.g. in the publication WO2017/182706 that blocking of Clever-1 turns alternatively activated macrophages from an immunosuppressive (M2) phenotype to a pro-inflammatory (M1) phenotype.

The publication WO 03/057130 also discloses that Clever-1 mediates binding of other types of leukocytes such as monocytes and granulocytes to HEV-like vessels. Thus, by blocking the interaction of Clever-1 and malignant tumour cells it became possible to control metastasis by preventing malignant cells that bind to Clever-1 from being taken up by the lymphatic vessels, and thus to prevent spread of the malignancy into the lymph nodes. Toll-like receptors (TLRs) are in generally present on many cells of the immune system and they have been shown to be involved in the innate immune response. Toll-like receptor 9 (TLR9) is a member of the toll-like receptor (TLR) family. TLR9 is an important receptor expressed in immune system cells including dendritic cells, macrophages, natural killer cells, and other antigen presenting cells. TLR9 preferentially binds DNA present in bacteria and viruses, and triggers signalling cascades that lead to a pro-inflammatory cytokine response. Cancer, infection, and tissue damage can all modulate TLR9 expression and activation. TLR9 agonists are presented to be potential for the treatment of cancer and several studies demonstrating antitumor activity in a wide variety of tumour models. TLR 9 agonists for cancer therapy are reviewed e.g. by Krieg A M (2008), Oncogene 27, 161-167, “Toll-like receptor 9 (TLR9) agonists in the treatment of cancer”.

### SUMMARY OF THE INVENTION

Now, the inventors have surprisingly discovered that a Toll-like receptor 9 (TLR9) agonists can be used to downregulate the expression of Clever-1 on alternatively activated macrophages. TLR9 agonist was found to reduce rapidly Clever-1 expression. The finding of the present invention shows that TLR9 agonists can be used as Clever-1 inhibitors in a method of treating diseases or conditions with alternatively activated macrophage profile, wherein TLR9 agonists are capable of downregulating Clever-1 on said macrophages. Alternatively activated macrophages are a class of the macrophages which are especially present in high numbers in the tumours, but also in chronic infections and therefore the finding of the present invention shows that TLR9 agonists can be used as Clever-1 inhibitors in the treatment of cancers and/or chronic infections in an individual.

An object of the present invention is to provide a method for affecting the immune system of an individual and a method of treating diseases or conditions related to the function of the immune system, said methods comprising downregulating the Clever-1 receptor on alternatively activated macrophages in said individual. Especially, an object of the present invention is to provide a method of treating cancer and/or chronic infection in an individual by downregulating Clever-1 expression on alternatively activated macrophages.

In order to achieve among others the objects presented above, the invention is characterized by what is presented in the enclosed independent claim. Some preferred embodiments of the invention will be described in the other claims.

The embodiments and advantages mentioned in this text relate, where applicable, both to the composition, the method as well as to the uses according to the invention, even though it is not always specifically mentioned.

The present invention concerns the use of a composition comprising a Toll-like receptor 9 (TLR9) agonist for the downregulation of Clever-1 for the reprogramming of alternatively activated immunosuppressive macrophages. The present invention concerns a composition comprising TLR9 agonist for use in a method of treating cancer in an individual by downregulating Clever-1 expression on alternatively activated immunosuppressive macrophages, and/or in a method of treating chronic infection in an individual by downregulating Clever-1 expression on alternatively activated immunosuppressive macrophages.

The present invention concerns a composition comprising a Toll-like receptor 9 (TLR9) agonist for use in a method of treating cancer and/or chronic infection, wherein an individual having alternatively activated immunosuppressive macrophages characterized by Clever-1 expression. According to an embodiment, the present invention concerns a composition comprising a Toll-like receptor 9 (TLR9) agonist for use in the treatment of cancer in an individual having diagnosed with tumour characterized by macrophages expressing Clever-1, which macrophages may be alternatively activated immunosuppressive macrophages and/or tumour associated macrophages characterized by Clever-1 expression. In another embodiment, the present invention concerns a composition comprising a Toll-like receptor 9 (TLR9) agonist for use in a method of treating chronic infection, wherein an individual having diagnosed with a granuloma of a latent infection characterized by macrophages expressing Clever-1.

A composition according to the present invention may comprise one or more TLR9 agonists. Especially, it has been observed that TLR9 agonist comprising CpG ODN downregulates Clever-1 expression on alternatively activated immunosuppressive macrophages.

The invention provides methods of inducing an immune response to cancer and/or chronic infection comprising administering to a patient one or more TLR9 agonists.

Blocking of Clever-1 on tumour associated macrophages by Clever-1 specific antibodies is previously shown in the publication WO2010/122217 to prevent metastasis; and/or reduce the size of malignant tumour; and/or reduce malignant tumour growth. Therefore, the finding of the present invention is also assumed to prevent metastasis; and/or reduce the size of malignant; and/or reduce malignant tumour growth by downregulating Clever-1.

According to one aspect, the present invention concerns a method for treating cancer or chronic infection patients, which method comprises downregulation of Clever-1 expression on immunosuppressive alternatively activated macrophages. According to one aspect, the present invention concerns a method for treating cancer patients, in which method Clever-1 positive macrophages in tumours and/or Clever-1 positive monocytes in the blood are screened from the patient prior treatment for improving therapy success, and then the composition comprising one or more TLR9 agonists are administered to said patient having diagnosed with said macrophages and/or monocytes characterized by Clever-1 expression.

In some embodiments, the use of one or more TLR9 agonists for the treatment of a cancer further comprises administering one or more additional anticancer agents, such as e.g. Clever-1 specific antibodies or fragments thereof.

### BRIEF DESCRIPTION OF THE DRAWINGS

FIG. 1 shows the results of Example, in which Clever-1 expression on macrophages was analysed after the polarized macrophages were stimulated with different TLR ligands, and

FIG. 2 shows the ratio of Clever-1 with CD163 or CD68 positive macrophages per tissue microarray (TMA) spot in different carcinomas.

### DETAILED DESCRIPTION OF THE INVENTION

The term “treatment” or “treating” shall be understood to include complete curing of a disease or disorder, as well as amelioration or alleviation of said disease or disorder.

The term “therapeutically effective amount” is meant to include any amount of a composition according to the present invention that is sufficient to bring about a desired therapeutic result.

The term “TLR9 agonist” or “TLR targeting ligand” generally refers to an oligonucleotide-based compound that is able to bind to a TLR9 receptor and to enhance, induce or modulate an immune stimulation mediated by TLR9.

Macrophages are traditionally divided into two distinct phenotypes: M1 and M2 macrophages. M1 macrophages are classical pro-inflammatory macrophages, which produce large quantities of pro-inflammatory cytokines and co-stimulatory molecules and are very efficient in activation of T-cell responses. M2 macrophages, in contrast, are immune suppressing cells, which synthesize anti-inflammatory cytokines and induce regulatory T cells and hence profoundly dampen antigen-driven T cell activation. Tumour-associated macrophages (TAMs) are considered harmful as they mature into M2 macrophages (tumour promoting macrophages) within the tumour environment and suppress anti-tumour immune response and mediate angiogenic switch, a crucial step in cancer growth. In the present description, the term “alternatively activated immunosuppressive macrophages” or “alternatively activated macrophages” is used to refer to macrophages which display an immunosuppressive (M2) phenotype, i.e. also called as M2 macrophages. Alternatively activated macrophages are increased in response to many infections. Alternatively activated macrophages, also referred as tumour associated macrophages (TAMs) are present in high numbers in tumours.

According to the present invention, a compound comprising one or more TLR9 agonist is used to downregulate Clever-1 expression on alternatively activated immunosuppressive macrophages for the treatment of cancer and/or chronic infection. Preferably, a compound comprising one or more TLR9 agonist is used to downregulate Clever-1 expression on alternatively activated immunosuppressive macrophages for the treatment of cancer and/or chronic infection that is screened to have a high level of Clever-1 positive tissue bound macrophages or circulating monocytes. According to an embodiment of the invention TLR9 agonist comprises one or more oligonucleotide-based agonists of TLR9.

Oligonucleotide based TLR9 agonist may be artificial DNA oligonucleotides containing the CpG motif. Toll-like receptor 9 (TLR9) senses unmethylated CpG dinucleotides, a hallmark of microbial DNA, that can be mimicked by synthetic oligonucleotides containing CpG motifs (CpG ODNs). Therefore, the TLR9 agonist may be an unmethylated CpG oligodeoxynucleotide, or an analogue or derivative thereof that is capable of binging TLR9. CpG oligodeoxynucleotides (or CpG ODN) are short single-stranded synthetic DNA molecules that contain unmethylated CpG dinucleotides in particular sequence contexts (CpG motifs). CpG ODNs contain a cytosine triphosphate deoxynucleotide (“C”) followed by a guanine triphosphate deoxynucleotide (“G”). The “p” refers to the phosphodiester link between consecutive nucleotides. CpG ODNs possess a partially or completely phosphorothioated (PS) backbone, as opposed to the natural phosphodiester (PO) backbone found in genomic bacterial DNA. According to an embodiment of the present invention TLR9 agonist comprises an oligodeoxynucleotide containing cytosine-phosphate-guanosine (CpG) motifs (CpG ODNs).

According to an embodiment of the present invention pharmaceutical preparations of the compositions comprise also suitable pharmaceutically acceptable carriers comprising excipients and auxiliaries that facilitate processing of the active compounds into preparations that can be used pharmaceutically.

A method for treating a patient with the present invention comprises administration of a composition according to the invention. A method according to the present invention of inducing an immune response to cancer and/or chronic infection comprising administering a composition comprising one or more TLR9 agonists to a patient.

According to an embodiment of the present invention, one or more TLR9 agonists are administered to a cancer patient for inhibiting Clever-1 expression on alternatively activated immunosuppressive macrophages and/or tumour associated macrophages. Especially, TLR9 agonist treatment may be beneficial for the cancer patient having diagnosed with tumour characterized by Clever-1 expression. It has been observed that all tumours are not Clever-1 positive, i.e. all tumours do not comprise alternatively activated immunosuppressive macrophages and/or tumour associated macrophages expressing Clever-1 or the expression levels may differ significantly. Therefore, it is beneficial to screen patients for Clever-1 positivity prior to treatment for providing good treatment response. According to the preferred embodiment of the present invention, a compound comprising TLR9 agonist can be used in a treatment of tumour comprising alternatively activated immunosuppressive macrophages expressing Clever-1.

The method for treating or preventing cancer by reducing the size of malignant tumour; by reducing malignant tumour growth; and/or by inhibiting cancer cell transmigration and metastasis formation according to the present invention is applicable to all forms of cancers. Especially, the finding of the present invention is assumed to target all kind of cancers characterized by Clever-1 positive macrophages. The term “cancer” generally refers to, without limitation, any malignant growth or tumour caused by abnormal or uncontrolled cell proliferation and/or division. Cancers may occur in an individual and may arise in any and all tissues.

According to another embodiment of the present invention, one or more TLR9 agonists are administered to the patient with chronic infection for inhibiting Clever-1 expression on alternatively activated macrophages, especially when an individual having diagnosed with a granuloma of a latent infection characterized by macrophages expressing Clever-1.

For improving treatment success, a method for treating may firstly comprise screening and characterizing Clever-1 positive macrophages in tumours, granuloma of a chronic latent infection and/or circulating Clever-1 positive monocytes from a peripheral blood sample, and then the composition comprising one or more TLR9 agonists are administered to a patient having said macrophages and/or circulating monocytes characterized by Clever-1 expression. The screening may be carried out e.g. by assaying a biopsy sample from the patient for determining Clever-1 expression.

The pharmaceutical compositions according to the present invention can be administered by any means that achieve their intended purpose. For example, administration can be intravenous, intraarticular, intra-tumoural, intra-granulomal or subcutaneous. The dose chosen should be therapeutically effective with regard to the disease treated. According to an embodiment of the invention, TLR9 agonist is administered in a pharmaceutically effective amount.

In some embodiments, the use of TLR9 agonist for the treatment of a cancer further comprises administering one or more additional anticancer agents.

### EXPERIMENTAL

The following example illustrates the principles of the present invention and is not intended to limit the scope of the invention. The following Example 1 shows that TLR9 agonist (CpG ODN) downregulates Clever-1. Example 2 shows Clever-1 expression on macrophages in different human tumours.

### Example 1: Macrophage TLR Stimulation Assay

Wildtype C57BL/6N mice (n=4) were sacrificed and their femurs and tibias flushed with PBS using a 30G needle. Bone marrow cells were counted, resuspended to 1.0×106 cells/ml in macrophage medium (complete IMDM (10% fetal bovine serum and penicillin/streptomycin) supplemented with 20 ng/ml M-CSF (315-02, Peprotech)) and incubated in non-tissue-culture-treated plates at 37 ° C. for one week. Half the medium was replaced with fresh macrophage medium on day 4. The differentiated BMDMs were detached with 10 mM EDTA in PBS and plated in 96-well U-bottom plates at a density of 1.0×105 cells/well. Thereafter, the cells were polarized with 10 nM dexamethasone for 24 h, which induced Clever-1 expression in approximately 80% of macrophages. The polarized macrophages were stimulated with different TLR ligands for indicated time points as follows: 10 μg/mL of Poly(I:C) (InVivogen); 100 ng/mL of LPS (Sigma); 5 μM of CpG ODN (InVivogen); 10 μg/mL of 2′,3′-cGAMP (InVivogen). Clever-1 expression on macrophages was analyzed by flow cytometry from permeabilized cells using 9-11 anti-Clever-1 antibody directly conjugated with allophycocyanin (APC) made in house. The Clever-1-APC signal was normalized to an irrelevant isotype control (ratIgG2a-APC) and shown as percent of cells positive for Clever-1.

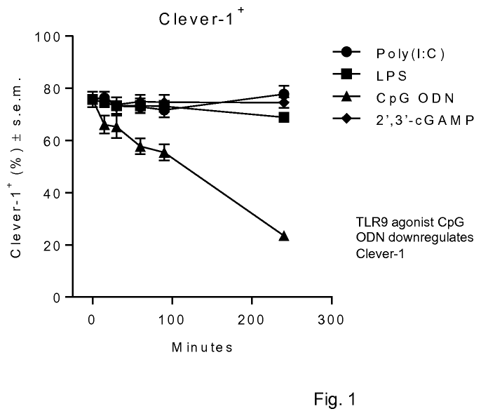

As seen in the FIG. 1, stimulation of alternatively activated macrophages with unmethylated CpG oligodeoxynucleotides (TLR9 agonist) induced rapid downregulation of Clever-1 expression by macrophages. A similar response was not seen with TLR3 (Poly(I:C)), TLR4 (LPS) or STING (2′,3′-cGAMP) agonists.

### Example 2: Clever-1 Expression on Macrophages in Human Tumours

Clever-1 expression on macrophages in different human tumors was studied.

Clever-1 expression was evaluated against the common macrophage marker CD68 (mouse anti-human CD68; Dako) and the M2 macrophage marker CD163 (mouse anti-human CD163; Thermo Fisher), and additionally the intensity of Clever-1 staining was evaluated. The number of positive cells for each marker (CD68, CD163 and Clever-1) was quantified from tissue microarray (TMA) spots. Tissue microarray (TMA) samples (formalin fixed paraffin embedded surgical primary tumor specimen) from different human carcinomas; glioma, breast, colorectal, gastric, lung, pancreatic, prostate and vulvar carcinoma were obtained from Auria Biobank (Turku, Finland).

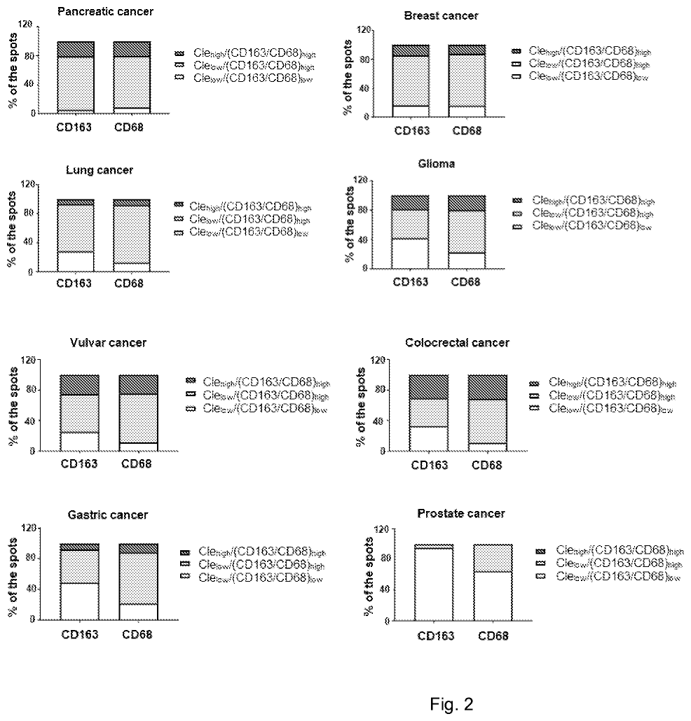

When comparing the ratio between Clever-1 high/low to CD163 or CD68 high/low TMA spots significant variation between cancer types was observed as shown in FIG. 2. FIG. 2 shows the ratio of Clever-1 with CD163 or CD68 positive macrophages per TMA spot in different carcinomas. The left bar indicates ratio with CD163 (for example Cle_{high}/CD163_{high}) and the right bar with CD68 (for example Cle_{high}/CD68_{high}). Classification of Clever-1: low=0-100 and high >100 positive cells, and of CD163/CD68 staining, low=0-200 and high >200 positive cells. The most abundant expression of Clever-1 was observed in samples from colorectal cancer, vulvar cancer, pancreatic cancer, gastric cancer, breast cancer and glioma, but not all patients in even these cancer types are Clever-1 positive. Prostate cancer on the other hand is extremely low in Clever-1 positive tumour associated macrophages, but individual variation may occur. Therefore, the screening of the patients for Clever-1 positivity prior to treatment provides possibility to select patients suitable for the treatment method targeting Clever-1 positive macrophages according to the present invention and may improve the treatment response.

1. A composition comprising a Toll-like receptor 9 (TLR9) agonist for use in a treatment of cancer and/or chronic infection in an individual, characterized in that said individual having alternatively activated immunosuppressive macrophages characterized by Clever-1 expression.

2. A composition comprising a Toll-like receptor 9 (TLR9) agonist for use in a treatment of cancer according to claim 1, characterized in that said individual having diagnosed with a tumour characterized by macrophages expressing Clever-1.

3. A composition comprising a Toll-like receptor 9 (TLR9) agonist for use in a treatment of chronic infection according to claim 1, characterized in that said Individual having diagnosed with a granuloma of a latent infection characterized by macrophages expressing Clever-1.

4. A composition comprising a Toll-like receptor 9 (TLR9) agonist for use in a treatment of cancer and/or chronic infection according to claim 1, characterized in that the TLR9 agonist comprises an unmethylated CPG oligodeoxynucleotide (CpG ODN).

5. A composition comprising a Toll-like receptor 9 (TLR9) agonist for use in a treatment of cancer and/or chronic infection according to claim 1 characterized in that TLR9 agonist is administered in a pharmaceutically effective amount.

6. A method of treating cancer and/or chronic infection in an individual comprising administering a pharmaceutically effective amount of a composition comprising a Toll-like receptor 9 (TLR9) agonist to the individual, wherein the individual has alternatively activated immunosuppressive macrophages characterized by Clever-1 expression.

7. The method of claim 6, wherein the method is treating cancer and wherein the individual has been diagnosed with a tumour characterized by macrophages expressing Clever-1.

8. The method of claim 6, wherein the method is treating chronic infection and wherein the individual has been diagnosed with a granuloma of a latent infection characterized by macrophages expressing Clever-1.

9. The method of claim 6, wherein the TLR9 agonist comprises an unmethylated CPG oligodeoxynucleotide (CpG ODN).

10. The method of claim 7, wherein the TLR9 agonist comprises an unmethylated CPG oligodeoxynucleotide (CpG ODN).

11. The method of claim 8, wherein the TLR9 agonist comprises an unmethylated CPG oligodeoxynucleotide (CpG ODN).
